(19) United States
(12) Patent Application Publication
(10) Pub. No.: US 2024/0352395 A1
(43) Pub. Date: Oct. 24, 2024

(54) Three-Dimensional Printing of Organs, Organoids, and Chimeric Immuno-Evasive Organs

(71) Applicant: Creative Medical Technologies, Inc., Phoenix, AZ (US)

(72) Inventors: Thomas Ichim, San Diego, CA (US); Timothy Warbington, Phoenix, AZ (US); Amit Patel, Salt Lake City, UT (US); Courtney Bartlett, Niceville, FL (US)

(73) Assignee: Creative Medical Technologies, Inc., Phoenix, AZ (US)

(21) Appl. No.: 18/636,179

(22) Filed: Apr. 15, 2024

Related U.S. Application Data
(60) Provisional application No. 63/460,543, filed on Apr. 19, 2023.

(51) Int. Cl. C12M 1/26; B33Y 10/00; B33Y 50/02; B33Y 70/10

(52) U.S. Cl. CPC C12M 33/04; B33Y 10/00; B33Y 50/02; B33Y 70/10

(57) ABSTRACT

Disclosed are means of generating organs or organoids utilizing three-dimensional bio-printing processes. In one embodiment said organ is a pancreas or pancreas-like organ useful for the treatment of diabetes, wherein said bioprinted pancreas possesses enhanced regenerative activity, in some embodiments comparable to the liver due to seeding with enhanced numbers of regenerative cells. In another embodiment pancreatic islets are created capable of being utilized as a substitute for naturally occurring islets. In other embodiments immune-evasive organs are created utilizing combination of cells which possess ability to evade immunity, thus allowing for tolerance induction without need for immune suppressive activity.

### CROSS-REFERENCE TO RELATED APPLICATIONS

The present application claims benefit of Provisional Patent Application Ser. No. 63/460,543, filed on Apr. 19, 2023, titled THREE-DIMENSIONAL PRINTING OF ORGANS, ORGANOIDS, AND CHIMERIC IMMUNO-EVASIVE ORGANS, the contents of which are incorporated herein by reference in its entirety.

### FIELD OF THE INVENTION

The invention belongs to the field of therapeutic tissue engineering, more specifically, the field belongs to the field of enhancing efficacy of transplanted therapeutic organs through selective seeding with regenerative cells and/or immune evasive cells and/or molecules.

### BACKGROUND OF THE INVENTION

It is estimated that Type 1 diabetes (T1D) targets 78,000 children annually worldwide. T1D, if it is left untreated, will lead to death of the patient. The diagnosis of T1D is made when the pancreas is producing very little, if any, insulin.

In some cases, the diagnosis is made by administering the glycated hemoglobin (A1C) test indicating a patient's average blood sugar level for the past two to three months. Insulin is a hormone that regulates blood glucose levels in the bodies' cells. Without insulin being present, glucose cannot enter the cells, which results in insufficient energy for the cells and ultimately cell death. The cells responsible for producing insulin are beta-cells, which, in the case of diabetic patients, are destroyed by the body's immune response system.

Today's most common treatment for T1D is injecting insulin by needle or pump, which is typically required before every meal (3-6 times/day), Additional injections (e.g., 1-2 times/day) might be necessary. This type of treatment, however, is far from optimal, because it leads to fluctuating glucose levels, which often lead to complications such as an increased risk of cardiovascular diseases and nerve, kidney, eye or foot damage. This is a result from diabetes ketoacidosis, which is caused by shortage of insulin, also called hyperglycemia. Toxic products form and collect because of the low pH level in the blood, which will ultimately be fatal if not adequately treated. Another severe complication that diabetic patients face is diabetic coma, which is due to shortage of glucose in the brain, called prolonged hypoglycemia. It leads to brain damage and possibly to death. Moreover, injecting by needle creates problems with patient compliance.

Another form of treatment which is relatively new in the field, islet transplantation, has been performed in a select group of patients with T1D. Different approaches have recently been evaluated by encapsulating islets in hydrogels Hydrogels have been used for cell encapsulation and in a variety of applications for tissue engineering. Hydrogel encapsulation has also been used for immune-protection of the encapsulated cell.

Studies indicate that islets of Langerhans transplantation and stem cell therapy, which result in beta-cell production, show promise for future treatment of T1D. However, a major challenge is the need to predict and monitor how a patient's immune system will react to such treatment, especially in the initial stages of the treatment.

The current invention provides means of bioengineering artificial organs in general and specifically pancreas and pancreas hybrids to overcome issues of rejection and possess enhanced regenerative features.

### SUMMARY OF THE INVENTION

Preferred methods are directed to embodiments wherein computer-implemented method for identifying agents capable of inducing specific differentiation of pluripotent stem cells into a particular tissue through the utilization of a computing system, said comprising: a) obtaining information regarding cellular and molecular characteristics of an embryonic

D Preferred embodiments include methods of bioprinting a three-dimensional organ comprising the steps of: a) obtaining a computer representation of said organ; b) identifying cellular populations comprising said organ utilizing a visualization and/or immunological visualization means; c) utilizing a system for sequentially layer cells upon each other in order to replication said organ needed to be replicated; and d) optionally growing said organ in vitro and/or in vivo.

Preferred methods include embodiments wherein said identification of said cellular populations is performed by histological analysis.

Preferred methods include embodiments wherein said identification of said cellular populations is performed by immunohistochemical analysis.

Preferred methods include embodiments wherein said identification of said cellular populations is performed by flow cytometric analysis.

Preferred methods include embodiments wherein said identification of said cellular populations is performed by mass cytometry analysis.

Preferred methods include embodiments wherein said identification of said cellular populations is performed by intracellular cytokine staining analysis.

Preferred methods include embodiments wherein said identification of said cellular populations is performed by analysis of transcription factors associated with particular cells.

Preferred methods include embodiments wherein said histochemical analysis is performed using one more staining techniques which allow for identification of various cellular properties.

Preferred methods include embodiments wherein said staining techniques involves application of hemoxylin staining.

Preferred methods include embodiments wherein said staining techniques involves application of eosinophilic staining.

Preferred methods include embodiments wherein said staining techniques involves application of nissle staining.

Preferred methods include embodiments wherein said staining techniques involves application of iodine staining.

Preferred methods include embodiments wherein said staining techniques involves application of methyl blue staining.

Preferred methods include embodiments wherein said staining techniques involves application of hemoxylin staining.

Preferred methods include embodiments wherein said histochemical analysis is performed using a computer assisted visualization means which incorporates principal component analysis.

Preferred methods include embodiments wherein said principal component analysis is performed by a deep learning system.

Preferred methods include embodiments wherein said deep learning said deep learning system is performed in a supervised manner.

Preferred methods include embodiments wherein said deep learning said deep learning system is performed in a unsupervised manner.

Preferred methods include embodiments wherein deep learning system is based on previous structural data provided by internal and/or third-party databases.

Preferred methods include embodiments wherein said deep learning is programed to identify and exclude organ abnormalities.

Preferred methods include embodiments wherein cells to be seeded into said organ are identified based on cell surface markers.

Preferred methods include embodiments wherein cells to be seeded into said organ are identified based on cell morphology.

Preferred methods include embodiments wherein cells to be seeded into said organ are identified based on identity associated homeobox protein expression.

Preferred methods include embodiments wherein cells to be seeded into said organ are identified based on molecular pathway analysis.

Preferred methods include embodiments wherein said molecular pathway analysis is based on expression of VEGF associated genes.

Preferred methods include embodiments wherein said molecular pathway analysis is based on expression of wnt associated genes.

Preferred methods include embodiments wherein said molecular pathway analysis is based on expression of notch associated genes.

Preferred methods include embodiments wherein said molecular pathway analysis is based on expression of jagged associated genes.

Preferred methods include embodiments wherein said molecular pathway analysis is based on expression of frizzled associated genes.

Preferred methods include embodiments wherein said molecular pathway analysis is based on expression of GDF-15 associated genes.

Preferred methods include embodiments wherein said molecular pathway analysis is based on expression of homeobox associated genes.

Preferred methods include embodiments wherein said molecular pathway analysis is based on expression of isl associated genes.

Preferred methods include embodiments wherein said molecular pathway analysis is based on expression of PDX-1 associated genes.

Preferred methods include embodiments wherein said molecular pathway analysis is based on expression of histone deacetylase associated genes.

Preferred methods include embodiments wherein said histone deacetylase is HDAC1.

Preferred methods include embodiments wherein said histone deacetylase is HDAC3.

Preferred methods include embodiments wherein said histone deacetylase is HDAC6.

Preferred methods include embodiments wherein said histone deacetylase is HDAC7.

Preferred methods include embodiments wherein said histone deacetylase is HDAC9.

Preferred methods include embodiments wherein said cells in said organ are identified based on endodermal, ectodermal and mesodermal content.

Preferred methods include embodiments wherein said bioprinting is performed in a media capable of sustaining cellular viability and activity.

Preferred methods include embodiments wherein said media is RPMI-1640 media.

Preferred methods include embodiments wherein said media is DMEM media.

Preferred methods include embodiments wherein said media is EMEM media.

Preferred methods include embodiments wherein said media is alpha MEM media.

Preferred methods include embodiments wherein said media is Iscove's media.

Preferred methods include embodiments wherein said media is AIM-V media.

Preferred methods include embodiments wherein said media is supplemented with platelet rich plasma.

Preferred methods include embodiments wherein said media is supplemented with platelet rich fibrin.

Preferred methods include embodiments wherein said media is supplemented with stem cell conditioned media.

Preferred methods include embodiments wherein said stem cell conditioned media is generated by culturing of stem cells in a liquid media.

Preferred methods include embodiments wherein said stem cell is a hematopoietic stem cell.

Preferred methods include embodiments wherein said stem cell is a mesenchymal stem cell.

Preferred methods include embodiments wherein said stem cell is a pluripotent stem cell.

Preferred methods include embodiments wherein said hematopoietic stem cell is capable of generating one or more lineages of blood cells.

Preferred methods include embodiments wherein said hematopoietic stem cell is plastic non-adherent.

Preferred methods include embodiments wherein said hematopoietic stem cell is adherent to bone marrow stroma.

Preferred methods include embodiments wherein said hematopoietic stem cell binds to said bone marrow stroma via LFA-1.

Preferred methods include embodiments wherein said hematopoietic stem cell binds to said bone marrow stroma via ICAM-1.

Preferred methods include embodiments wherein said hematopoietic stem cell binds to said bone marrow stroma via PECAM-1.

Preferred methods include embodiments wherein said hematopoietic stem cell binds to said bone marrow stroma via VLA-4.

Preferred methods include embodiments wherein said hematopoietic stem cell binds to said bone marrow stroma via an alpha v integrin.

Preferred methods include embodiments wherein said alpha v integrin is alpha v beta 3.

Preferred methods include embodiments wherein said hematopoietic stem cell expresses fucosylated antigens.

Preferred methods include embodiments wherein said hematopoietic stem cell proliferates in response to interleukin-3.

Preferred methods include embodiments wherein said hematopoietic stem cell proliferates in response to interleukin-6.

Preferred methods include embodiments wherein said hematopoietic stem cell proliferates in response to M-CSF.

Preferred methods include embodiments wherein said hematopoietic stem cell proliferates in response to GM-CSF.

Preferred methods include embodiments wherein said hematopoietic stem cell proliferates in response to angiopoietin.

Preferred methods include embodiments wherein said hematopoietic stem cell proliferates in response to steel factor.

Preferred methods include embodiments wherein said hematopoietic stem cell possesses reduced proliferation in the presence of TGF-beta.

Preferred methods include embodiments wherein said hematopoietic stem cell possesses reduced proliferation in the presence of endoglin.

Preferred methods include embodiments wherein said hematopoietic stem cell possesses reduced proliferation in the presence of angiostatin.

Preferred methods include embodiments wherein said hematopoietic stem cell possesses reduced proliferation in the presence of interleukin-10.

Preferred methods include embodiments wherein said hematopoietic stem cell possesses an enhanced proclivity to differentiation into the thrombocytic lineage when exposed to TPO.

Preferred methods include embodiments wherein said hematopoietic stem cell expresses interleukin-3 receptor.

Preferred methods include embodiments wherein said hematopoietic stem cell expresses HGF receptor.

Preferred methods include embodiments wherein said hematopoietic stem cell expresses acidic FGF receptor.

Preferred methods include embodiments wherein said hematopoietic stem cell expresses basic FGF receptor.

Preferred methods include embodiments wherein said hematopoietic stem cell expresses platelet derived growth factor receptor.

Preferred methods include embodiments wherein said hematopoietic stem cell expresses endoglin receptor.

Preferred methods include embodiments wherein said hematopoietic stem cell expresses substance P receptor.

Preferred methods include embodiments wherein said hematopoietic stem cell expresses CD33.

Preferred methods include embodiments wherein said hematopoietic stem cell expresses CD34.

Preferred methods include embodiments wherein said hematopoietic stem cell expresses CD133.

Preferred methods include embodiments wherein said hematopoietic stem cell expresses c-kit.

Preferred methods include embodiments wherein said hematopoietic stem cell expresses CD56.

Preferred methods include embodiments wherein said hematopoietic stem cell expresses leukemia inhibitory factor receptor.

Preferred methods include embodiments wherein said hematopoietic stem cell expresses complement C5 receptor.

Preferred methods include embodiments wherein said hematopoietic stem cell expresses complement C3 receptor.

Preferred methods include embodiments wherein said hematopoietic stem cell expresses CXCR4.

Preferred methods include embodiments wherein said hematopoietic stem cell increases expression of CXCR4 under hypoxia.

Preferred methods include embodiments wherein said hematopoietic stem cell migrates towards stromal derived factor-1.

Preferred methods include embodiments wherein said hematopoietic stem cell migrates towards agonists of toll like receptor 4.

Preferred methods include embodiments wherein said hematopoietic stem cell proliferates in response to ligation of toll like receptor 4.

Preferred methods include embodiments wherein said hematopoietic stem cell secretes interleukin-1 in response to ligation of toll like receptor 4.

Preferred methods include embodiments wherein said hematopoietic stem cell proliferates in response to ligation of toll like receptor 2.

Preferred methods include embodiments wherein said hematopoietic stem cell proliferates in response to ligation of toll like receptor 9.

Preferred methods include embodiments wherein said agent capable of ligating said toll like receptor 4 is an antibody.

Preferred methods include embodiments wherein said agent capable of ligating said toll like receptor 4 is an IgG antibody.

Preferred methods include embodiments wherein said agent capable of ligating said toll like receptor 4 is an IgG2b antibody.

Preferred methods include embodiments wherein said agent capable of ligating said toll like receptor 4 is an IgG1 antibody.

Preferred methods include embodiments wherein said agent capable of ligating said toll like receptor 4 is a bispecific antibody.

Preferred methods include embodiments wherein said agent capable of ligating said toll like receptor 4 is an asymmetric antibody.

Preferred methods include embodiments wherein said agent capable of ligating said toll like receptor 4 is a cameloid antibody.

Preferred methods include embodiments wherein said agent capable of ligating said toll like receptor 4 is a non-complement fixing antibody.

Preferred methods include embodiments wherein said agent capable of ligating said toll like receptor 4 is a IgM antibody.

Preferred methods include embodiments wherein said agent capable of ligating said toll like receptor 4 a danger associated molecular pattern.

Preferred methods include embodiments Preferred methods include embodiments wherein said danger associated molecular pattern is lipopolysaccharide.

Preferred methods include embodiments wherein said danger associated molecular pattern is HMGB-1.

Preferred methods include embodiments wherein said danger associated molecular pattern is a peptide of HMGB-1.

Preferred methods include embodiments wherein said danger associated molecular pattern is a E coli membrane lipophilic extract.

Preferred methods include embodiments wherein said danger associated molecular pattern is beta glucan.

Preferred methods include embodiments wherein said agent capable of ligating said toll like receptor 4 is morphine.

Preferred methods include embodiments wherein said agent capable of ligating said toll like receptor 4 is hsp-96

Preferred methods include embodiments wherein said hematopoietic stem cell proliferates in response to ligation of toll like receptor 5.

Preferred methods include embodiments wherein said agent capable of ligating said toll like receptor 5 is flagellin.

Preferred methods include embodiments wherein said agent capable of ligating said toll like receptor 5 is a flagellin peptide.

Preferred methods include embodiments wherein said organ is bioprinted in a manner such that endothelial cells are seeded as endothelial progenitor cells.

Preferred methods include embodiments wherein said endothelial progenitor cells are seeded approximately 1 nm to 5 mm apart from each other.

Preferred methods include embodiments wherein said endothelial progenitor cells are seeded approximately 5 nm to 3 mm apart from each other.

Preferred methods include embodiments wherein said endothelial progenitor cells are seeded approximately 10 nm to 1 mm apart from each other.

Preferred methods include embodiments wherein said endothelial progenitor cells are seeded approximately 100 nm to 1 mm apart from each other.

Preferred methods include embodiments wherein said endothelial progenitor cells are seeded approximately 1 micrometer apart from each other.

Preferred methods include embodiments wherein said endothelial progenitor cells are seeded only an extracellular matrix that has been deposited by the bioprinting device.

Preferred methods include embodiments wherein said extracellular matrix is a collagen.

Preferred methods include embodiments wherein said collagen is type 1 collagen.

Preferred methods include embodiments wherein said collagen is type 2 collagen.

Preferred methods include embodiments wherein said collagen is type 3 collagen.

Preferred methods include embodiments wherein said collagen is type 5 collagen.

Preferred methods include embodiments wherein said collagen is type 7 collagen.

Preferred methods include embodiments wherein said collagen is type 9 collagen.

Preferred methods include embodiments wherein said collagen is type 13 collagen.

Preferred methods include embodiments wherein said collagen is a mixture of collagens.

Preferred methods include embodiments wherein said extracellular matrix is a hyaluronic acid.

Preferred methods include embodiments wherein said hyaluronic acid is high molecular weight hyaluronic acid.

Preferred methods include embodiments wherein said high molecular weight hyaluronic acid is higher than 10 kDa in size.

Preferred methods include embodiments wherein said high molecular weight hyaluronic acid is higher than 50 kDa in size.

Preferred methods include embodiments wherein said high molecular weight hyaluronic acid is higher than 100 kDa in size.

Preferred methods include embodiments wherein said high molecular weight hyaluronic acid is higher than 10 kDa in size.

Preferred methods include embodiments wherein said extracellular matrix is one or more glycosaminoglycans.

Preferred methods include embodiments wherein said glycosaminoglycan is seed with extracellular matrix binding plasma derived factors.

Preferred methods include embodiments wherein said plasma derived factors are isolated from peripheral blood plasma.

Preferred methods include embodiments wherein said plasma derived factors are isolated from umbilical cord blood plasma.

Preferred methods include embodiments wherein said plasma derived factors are isolated from peripheral blood plasma from a patient receiving stem cell mobilization therapy.

Preferred methods include embodiments wherein said stem cell mobilization therapy involves entry of CD34 cells into systemic circulation.

Preferred methods include embodiments wherein said stem cell mobilization therapy is treated with on or more agents that reduces CXCR4 on CD34 cells.

Preferred methods include embodiments wherein said agent that reduces CXCR4 is a small molecular SDF-1 antagonist.

Preferred methods include embodiments wherein said small molecule SDF-1 antagonist is mozibil.

Preferred methods include embodiments wherein said agent that reduces CXCR4 is G-CSF.

Preferred methods include embodiments wherein said agent that reduces CXCR4 is GM-CSF.

Preferred methods include embodiments wherein said agent that reduces CXCR4 is flt-3 ligand.

Preferred methods include embodiments wherein said agent that reduces CXCR4 is HMGB1.

Preferred methods include embodiments wherein said agent that reduces CXCR4 is antisense DNA molecules to CXCR4.

Preferred methods include embodiments wherein said agent that reduces CXCR4 is short interfering RNA capable of inducing RNA interference to the CXCR4 mRNA.

Preferred methods include embodiments wherein said agent that reduces CXCR4 is short hairpin RNA capable of inducing RNA interference to the CXCR4 mRNA.

Preferred methods include embodiments wherein said agent that reduces CXCR4 a CRIPSR-based gene editing procedure capable of gene editing the CXCR4 DNA so as to block and/or impair transcription of said CXCR4 gene.

Preferred methods include embodiments wherein said plasma derived growth factor is BDNF.

Preferred methods include embodiments wherein said plasma derived growth factor is albumin-bound BDNF.

Preferred methods include embodiments wherein said plasma derived growth factor is hyaluronic acid-bound BDNF.

Preferred methods include embodiments wherein said plasma derived growth factor is BDNF complexed to anti-BDNF antibody.

Preferred methods include embodiments wherein said plasma derived growth factor is BDNF admixed with IVIG.

Preferred methods include embodiments wherein said plasma derived growth factor is interleukin-22.

Preferred methods include embodiments wherein said plasma derived growth factor is hepatocyte growth factor.

Preferred methods include embodiments wherein said hepatocyte growth factor is admixed with plasma.

Preferred methods include embodiments wherein said hepatocyte growth factor is admixed with albumin.

Preferred methods include embodiments wherein said hepatocyte growth factor is admixed with collagen II.

Preferred methods include embodiments wherein said hepatocyte growth factor is admixed with hyaluronic acid.

Preferred methods include embodiments wherein said hepatocyte growth factor is admixed with matrigel.

Preferred methods include embodiments wherein said hepatocyte growth factor is admixed with lipoprotein related receptor-1.

Preferred methods include embodiments wherein said hepatocyte growth factor is admixed with glycosaminoglycans.

Preferred methods include embodiments wherein said hepatocyte growth factor is admixed syndecan.

Preferred methods include embodiments wherein said hepatocyte growth factor is admixed with tissue inhibitor of matrix metalloprotease-1.

Preferred methods include embodiments wherein said hepatocyte growth factor is admixed with tissue inhibitor of matrix metalloprotease-3.

Preferred methods include embodiments wherein said hepatocyte growth factor is admixed with tissue inhibitor of matrix metalloprotease-7.

Preferred methods include embodiments wherein said hepatocyte growth factor is admixed with tissue inhibitor of matrix metalloprotease-9.

Preferred methods include embodiments wherein said hepatocyte growth factor is admixed with tissue inhibitor of matrix metalloprotease-13.

Preferred methods include embodiments wherein said hepatocyte growth factor is admixed with decellularized tissue matrix.

Preferred methods include embodiments, wherein said decellularized tissue matrix is obtained from a tissue associated with regeneration.

Preferred methods include embodiments wherein said tissue associated with regeneration is a tissue enriched for progenitor cells.

Preferred methods include embodiments wherein said tissue for progenitor cells possesses higher numbers of progenitor cells as compared to surrounding tissues.

Preferred methods include embodiments wherein said progenitor cells express aldehyde dehydrogenase.

Preferred methods include embodiments wherein said progenitor cells express CD133.

Preferred methods include embodiments wherein said progenitor cells express ability to efflux rhodamine 132.

Preferred methods include embodiments wherein said progenitor cells interleukin-22 receptor.

Preferred methods include embodiments wherein said progenitor cells express stem cell factor receptor.

Preferred methods include embodiments wherein said tissue associated with enhanced progenitor cells is testicular tissue.

Preferred methods include embodiments wherein said tissue associated with enhanced progenitor cells is ovarian tissue.

Preferred methods include embodiments wherein said tissue associated with enhanced progenitor cells is endometrial tissue.

Preferred methods include embodiments wherein said tissue associated with enhanced progenitor cells is placental tissue.

Preferred methods include embodiments wherein said placental tissue is derived from the hemochorial portion of said placenta.

Preferred methods include embodiments wherein said placental tissue is derived from the area in which fetal trophoblasts are invading maternal myometrium.

Preferred methods include embodiments wherein said tissue associated with enhanced progenitor cells is lymphatic tissue.

Preferred methods include embodiments wherein said tissue associated with enhanced progenitor cells is thymic tissue.

Preferred methods include embodiments wherein said tissue associated with enhanced progenitor cells is bone marrow tissue.

Preferred methods include embodiments wherein said tissue associated with enhanced progenitor cells is hippocampal tissue.

Preferred methods include embodiments wherein said tissue associated with enhanced progenitor cells is dentate gyrus tissue.

Preferred methods include embodiments wherein said tissue associated with enhanced progenitor cells is subventricular zone tissue.

Preferred methods include embodiments wherein said tissue associated with enhanced progenitor cells is splenic tissue.

Preferred methods include embodiments wherein said tissue associated with enhanced progenitor cells is keloid tissue.

Preferred methods include embodiments wherein said tissue associated with enhanced progenitor cells is regenerating liver tissue.

Preferred methods include embodiments wherein said tissue associated with enhanced progenitor cells is hair follicle tissue.

Preferred methods include embodiments wherein said tissue associated with enhanced progenitor cells is deciduous tooth tissue.

Preferred methods include embodiments wherein said tissue associated with enhanced progenitor cells is fallopian tube tissue.

Preferred methods include embodiments wherein said decellularized tissue is decellularized by treatment with a substance capable of liquifying cellular membranes while allowing integrity extracellular matrix architecture.

Preferred methods include embodiments wherein said liquifying agent is N-lauroylsarcosinate.

Preferred methods include embodiments wherein said liquifying agent is n-octyl-b-D-glucopyranoside.

Preferred methods include embodiments wherein said liquifying agent is polyoxyethylene alcohol.

Preferred methods include embodiments wherein said liquifying agent is polyoxyethylene isoalcohol.

Preferred methods include embodiments wherein said liquifying agent is polyoxyethylene p-t-octyl phenol.

Preferred methods include embodiments wherein said liquifying agent is one or more polyoxyethylene esters of a fatty acid.

Preferred methods include embodiments wherein said mesenchymal stem cell is plastic adherent.

Preferred methods include embodiments wherein said mesenchymal stem cell expresses CD90.

Preferred methods include embodiments wherein said mesenchymal stem cell expresses CD105.

Preferred methods include embodiments wherein said mesenchymal stem cell expresses a combination of CD90 and CD105.

Preferred methods include embodiments wherein said mesenchymal stem cell expresses higher concentration of CD90 than CD105.

Preferred methods include embodiments wherein said mesenchymal stem cell expresses CD73

Preferred methods include embodiments wherein said mesenchymal stem cell expresses higher concentrations of CD73 in response to toll like receptor 3 stimulation.

Preferred methods include embodiments wherein said toll like receptor 3 stimulation is achieved by exposure of said mesenchymal stem cell to double stranded RNA.

Preferred methods include embodiments wherein said toll like receptor 3 stimulation is achieved by exposure of said mesenchymal stem cell to Poly IC.

Preferred methods include embodiments wherein said toll like receptor 3 stimulation is achieved by exposure of said mesenchymal stem cell to Poly ICIC.

Preferred methods include embodiments wherein said mesenchymal stem cell expresses higher concentrations of PD-L1 in response to toll like receptor 4 stimulation.

Preferred methods include embodiments wherein said mesenchymal stem cell expresses higher concentrations of PD-L1 in response to toll like receptor 5 stimulation.

Preferred methods include embodiments wherein said mesenchymal stem cell expresses higher concentrations of PD-L1 in response to toll like receptor 7 stimulation.

Preferred methods include embodiments wherein said mesenchymal stem cell expresses higher concentrations of PD-L1 in response to toll like receptor 9 stimulation.

Preferred methods include embodiments wherein said TLR4 stimulator is lipopolysaccharide.

Preferred methods include embodiments wherein said TLR4 stimulator is low molecular weight hyaluronic acid.

Preferred methods include embodiments wherein said TLR4 stimulator is HMGB1.

Preferred methods include embodiments wherein said TLR4 stimulator is heat shock protein 96.

Preferred methods include embodiments wherein said TLR5 stimulator is flagellin.

Preferred methods include embodiments wherein said TLR7 stimulator is imiquimod.

Preferred methods include embodiments wherein said TLR stimulator is CpG DNA.

Preferred methods include embodiments wherein said cellular populations used for said bioprinting procedures are selected from a group comprising of: group consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, Littre gland cells, uterus endometrium cells, goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cells, oxyphil cells, adrenal gland cells, chromaffin cells, Leydig cells, theca interna cells, corpus luteum cells, granulosa lutein cells, theca lutein cells, juxtaglomerular cells, macula densa cells, peripolar cells, mesangial cells, blood vessel and lymphatic vascular endothelial fenestrated cells, blood vessel and lymphatic vascular endothelial continuous cells, blood vessel and lymphatic vascular endothelial splenic cells, synovial cells, peritoneal serosal cells, pleural serosal cells, pericardial cavity serosal cells, squamous cells, columnar cells, dark cells, vestibular membrane cells, stria vascularis basal cells, stria vascularis marginal cells, cells of Claudius, cells of Boettcher, choroid plexus cells, arachnoid squamous cells, pigmented ciliary epithelium cells, non-pigmented ciliary epithelium cells, corneal endothelial cells, peg cells, respiratory tract ciliated cells, oviduct ciliated cells, uterine endometrial ciliated cells, rete testis ciliated cells, ductulus efferens ciliated cells, ciliated ependymal cells, epidermal keratinocytes, epidermal basal cells, fingernail and toenail keratinocytes, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, stratified squamous epithelium, epithelial basal cells, urinary epithelium cells, inner auditory hair cells of the organ of Corti, outer auditory hair cells of the organ of Corti, basal cells of olfactory epithelium, cold-sensitive primary sensory neurons, heat-sensitive primary sensory neurons, epidermal Merkel cells, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cells, type I hair cell of the vestibular apparatus of the ear, type II hair cell of the vestibular apparatus of the ear, type I taste bud cells, cholinergic neural cells, adrenergic neural cells, peptidergic neural cells, inner pillar cells of the organ of Corti, outer pillar cells of the organ of Corti, inner phalangeal cells of the organ of Corti, outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tube cells, kidney collecting duct cells, type I pneumocytes, pancreatic duct cells, non-striated duct cells, duct cells, intestinal brush border cells, exocrine gland striated duct cells, gall bladder epithelial cells, ductus efferens non-ciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, non-epithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, cochlear stellate cells, hepatic stellate cells, pancreatic stellate cells, red skeletal muscle cells, white skeletal muscle cells, intermediate skeletal muscle cells, nuclear bag cells of the muscle spindle, nuclear chain cells of the muscle spindle, satellite cells, cardiomyocytes, nodal cardiomyocytes, Purkinje fiber cells, smooth muscle cells, myoepithelial cells of the iris, myoepithelial cells of the exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cells, natural killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, and intestinal kidney cells.

Preferred methods include embodiments wherein bioprinting comprises three-dimensional printing of a biological organ, organoid, and/or tissue through the layering of living cells using a bioprinter.

Preferred methods include embodiments wherein said bioprinter is a three-axis mechanical platform that controls the movements of extruders that deposit layers of living cells in a desired shape.

Preferred methods include embodiments wherein said desired shape is acquired by scanning the surface of a desired organ, organoid and/or tissue to generate a surface map for guidance with cell deposition.

Preferred methods include embodiments wherein scanning the surface of a desired organ, organoid and/or tissue is achieved using a laser, electron beam, magnetic resonance imaging, microwave, x-ray, computed tomography, or a combination thereof.

Preferred methods include embodiments wherein said three dimensional organ is manufactured in a manner to possess reduced immunogenicity as compared to a wild type organ.

Preferred methods include embodiments wherein said reduced immunogenicity is accomplished by inhibition of immunogenic epitopes.

Preferred methods include embodiments wherein said immunogenic epitopes are derived from molecules originating from the human leukocyte antigen (HLA) class of proteins.

Preferred methods include embodiments wherein said HLA is HLA-A.

Preferred methods include embodiments wherein said HLA is HLA-B.

Preferred methods include embodiments wherein said HLA is HLA-C.

Preferred methods include embodiments wherein said HLA is HLA-DR.

Preferred methods include embodiments wherein said HLA is HLA-DQ.

Preferred methods include embodiments wherein said HLA is HLA-DP.

Preferred methods include embodiments wherein said reduction in HLA expression is achieved by gene editing to remove said HLA molecules from cells to be manufactured as part of the bioprinted organ.

Preferred methods include embodiments wherein said gene editing is accomplished through the use of CRISPR mediated gene excision.

Preferred methods include embodiments wherein said gene editing is accomplished through the use of Cas9 mediated gene excision.

Preferred methods include embodiments wherein said reduction in HLA expression is achieved by utilizing an mRNA targeting approach in order to reduce production of HLA molecules from cells to be manufactured as part of the bioprinted organ.

Preferred methods include embodiments wherein said mRNA targeting approach is utilization of antisense oligonucleotides specific to one or more HLA molecules.

Preferred methods include embodiments wherein said antisense molecule is comprising of DNA complementary to the mRNA sequence.

Preferred methods include embodiments wherein said complementarity is sufficient to induce activation of the enzyme RNAse H.

Preferred methods include embodiments wherein said RNAse H activation is sufficient to induce degradation of the mRNA coding for one or more HLA molecules.

Preferred methods include embodiments wherein said antisense molecule is comprised of synthetic nucleotides.

Preferred methods include embodiments wherein said antisense molecule is chemically modified to induced enhanced stability.

Preferred methods include embodiments wherein said antisense molecule is chemically modified by sulfonation in order to endow enhanced stability.

Preferred methods include embodiments wherein said antisense oligonucleotide is delivered via the use of electroporation.

Preferred methods include embodiments wherein said antisense oligonucleotide is delivered via the use of cell penetrating peptide.

Preferred methods include embodiments wherein said cell penetrating peptide is LL-37.

Preferred methods include embodiments wherein said cell penetrating peptide is αgliadin (31-43).

Preferred methods include embodiments wherein said cell penetrating peptide is Alyteserin-2a.

Preferred methods include embodiments wherein said cell penetrating peptide is AT1AR (304-318).

Preferred methods include embodiments wherein said cell penetrating peptide is BPP13a.

Preferred methods include embodiments wherein said cell penetrating peptide is Buforin II.

Preferred methods include embodiments wherein said cell penetrating peptide is C105Y.

Preferred methods include embodiments wherein said cell penetrating peptide is CB5005 M.

Preferred methods include embodiments wherein said cell penetrating peptide is CGKRK.

Preferred methods include embodiments wherein said cell penetrating peptide is CIGB-300.

Preferred methods include embodiments wherein said cell penetrating peptide is CpMTP.

Preferred methods include embodiments wherein said cell penetrating peptide is CPPecp.

Preferred methods include embodiments wherein said cell penetrating peptide is DPV1047.

Preferred methods include embodiments wherein said cell penetrating peptide is DRTTLTN.

preferred methods include embodiments wherein said cell penetrating peptide is EPRNEEK.

Preferred methods include embodiments wherein said cell penetrating peptide is gH625.

Preferred methods include embodiments wherein said cell penetrating peptide is GV1001.

Preferred methods include embodiments wherein said cell penetrating peptide is HAIYPRH.

Preferred methods include embodiments wherein said cell penetrating peptide is I1WL5W.

Preferred methods include embodiments wherein said cell penetrating peptide is KAFAK.

Preferred methods include embodiments wherein said cell penetrating peptide is lactoferrampin (265-284).

Preferred methods include embodiments wherein said cell penetrating peptide is lactoferricin (17-30).

Preferred methods include embodiments wherein said cell penetrating peptide is LMWP.

Preferred methods include embodiments wherein said cell penetrating peptide is lycosin-I.

Preferred methods include embodiments wherein said cell penetrating peptide is Maurocalcine.

Preferred methods include embodiments wherein said cell penetrating peptide is MCoTI.

Preferred methods include embodiments wherein said cell penetrating peptide is Myr-ApoE.

Preferred methods include embodiments wherein said cell penetrating peptide is P14LRR.

Preferred methods include embodiments wherein said cell penetrating peptide is PenetraMax.

Preferred methods include embodiments wherein said cell penetrating peptide is Peptide 599.

Preferred methods include embodiments wherein said cell penetrating peptide is PepFect14.

Preferred methods include embodiments wherein said cell penetrating peptide is Pyrrhocoricin.

Preferred methods include embodiments wherein said cell penetrating peptide is R6dGR.

Preferred methods include embodiments wherein said cell penetrating peptide is R9-H4A2.

Preferred methods include embodiments wherein said cell penetrating peptide is R10W6.

Preferred methods include embodiments wherein said cell penetrating peptide is CRGDfC.

Preferred methods include embodiments wherein said cell penetrating peptide is RT53.

Preferred methods include embodiments wherein said cell penetrating peptide is RTP004.

Preferred methods include embodiments wherein said cell penetrating peptide is RVG29.

Preferred methods include embodiments wherein said cell penetrating peptide is RW16.

Preferred methods include embodiments wherein said cell penetrating peptide is vCPP 2319.

Preferred methods include embodiments wherein said reduction of immunogenic molecules is accomplished by administration of agents capable of inducing the process of RNA interference.

Preferred methods include embodiments wherein said RNA interference is induced by administration of short interfering RNA.

Preferred methods include embodiments wherein said RNA interference is induced by administration of a ribozyme.

Preferred methods include embodiments wherein said RNA interference is induced by administration of microRNA.

Preferred methods include embodiments wherein said RNA interference is induced by administration of short hairpin RNA.

Preferred methods include embodiments wherein said RNA interference is induced by administration of short interfering circular RNA.

Preferred methods include embodiments wherein said nucleic acids capable of inducing RNA interference are administered through the use of a cell penetrating peptide.

Preferred methods include embodiments wherein said reduction of immunogenicity of immunogenic epitopes is accomplished by engineering the organ with immune regulatory molecules.

Preferred methods include embodiments wherein said immune regulatory molecules are inducible only in the presence of an immune response.

Preferred methods include embodiments wherein said immune regulatory molecules are inducible only in the presence of an inflammatory response.

Preferred methods include embodiments wherein said immune regulatory molecules are inducible in the presence of interferon gamma.

Preferred methods include embodiments wherein said immune regulatory molecules are inducible in the presence of TNF-alpha.

Preferred methods include embodiments wherein said immune regulatory molecules are inducible in the presence of interleukin-6.

Preferred methods include embodiments wherein said immune regulatory molecules are inducible in the presence of lymphotoxin.

Preferred methods include embodiments wherein said immune regulatory molecules are inducible in the presence of MCP-1.

Preferred methods include embodiments wherein said immune regulatory molecules are inducible in the presence of MIP-1 alpha.

Preferred methods include embodiments wherein said immune regulatory molecules are inducible in the presence of MIP-1 beta.

Preferred methods include embodiments wherein said immune regulatory molecules are inducible in the presence of HMGB-1.

Preferred methods include embodiments wherein said immune regulatory molecules are inducible in the presence of complement C1.

Preferred methods include embodiments wherein said immune regulatory molecules are inducible in the presence of complement C3a.

Preferred methods include embodiments wherein said immune regulatory molecules are inducible in the presence of complement C3b.

Preferred methods include embodiments wherein said immune regulatory molecules are inducible in the presence of complement C5.

Preferred methods include embodiments wherein said immune regulatory molecules are inducible in the presence of complement membrane attack complex components.

Preferred methods include embodiments wherein said immune regulatory molecules are inducible in the presence of anaphylatoxin.

Preferred methods include embodiments wherein said immune regulatory molecules are inducible in the presence of interleukin-1 beta.

Preferred methods include embodiments wherein said immune regulatory molecules are inducible in the presence of interleukin-5.

Preferred methods include embodiments wherein said immune regulatory molecules are inducible in the presence of interleukin-8.

Preferred methods include embodiments wherein said immune regulatory molecules are inducible in the presence of interleukin-9.

Preferred methods include embodiments wherein said immune regulatory molecules are inducible in the presence of interleukin-11.

Preferred methods include embodiments wherein said immune regulatory molecules are inducible in the presence of interleukin-12.

Preferred methods include embodiments wherein said immune regulatory molecules are inducible in the presence of interleukin-15.

Preferred methods include embodiments wherein said immune regulatory molecules are inducible in the presence of interleukin-17.

Preferred methods include embodiments wherein said immune regulatory molecules are inducible in the presence of interleukin-18.

Preferred methods include embodiments wherein said immune regulatory molecules are inducible in the presence of interleukin-21.

Preferred methods include embodiments wherein said immune regulatory molecules are inducible in the presence of interleukin-23.

Preferred methods include embodiments wherein said immune regulatory molecules are inducible in the presence of interleukin-27.

Preferred methods include embodiments wherein said immune regulatory molecules are inducible in the presence of interleukin-33.

Preferred methods include embodiments wherein said immune regulatory molecule is inducible by AIRE.

Preferred methods include embodiments wherein said immune regulatory molecule is inducible by FoxP3.

Preferred methods include embodiments wherein said immune regulatory molecule is AIRE.

Preferred methods include embodiments wherein said immune regulatory molecule is FoxP3.

Preferred methods include embodiments wherein said immune regulatory molecule is interleukin 1 receptor antagonist.

Preferred methods include embodiments wherein said immune regulatory molecule is interleukin-12 p40 homodimer.

Preferred methods include embodiments wherein said immune regulatory molecule is interleukin-1 receptor antagonist.

Preferred methods include embodiments wherein said immune regulatory molecule is LAG-3.

Preferred methods include embodiments wherein said immune regulatory molecule is BlyS.

Preferred methods include embodiments wherein said immune regulatory molecule is soluble TNF receptor p55.

Preferred methods include embodiments wherein said immune regulatory molecule is soluble TNF receptor p75.

Preferred methods include embodiments wherein said immune regulatory molecule is soluble TRAIL receptor.

Preferred methods include embodiments wherein said immune regulatory molecule is soluble HLA-G.

Preferred methods include embodiments wherein said immune regulatory molecule is CTLA-4.

Preferred methods include embodiments wherein said immune regulatory molecule is PD-L1.

Preferred methods include embodiments wherein said immune regulatory molecule is PD-L2.

Preferred methods include embodiments wherein said immune regulatory molecule is arginase.

Preferred methods include embodiments wherein said immune regulatory molecule is prostaglandin E2

Preferred methods include embodiments wherein said immune regulatory molecule is galectin-1.

Preferred methods include embodiments wherein said immune regulatory molecule is galectin-3.

Preferred methods include embodiments wherein said immune regulatory molecule is galectin-7.

Preferred methods include embodiments wherein said immune regulatory molecule is galectin-9.

Preferred methods include embodiments wherein said immune regulatory molecule is TGF-beta.

Preferred methods include embodiments wherein said immune regulatory molecule is endoglin.

Preferred methods include embodiments wherein said immune regulatory molecule is angiopoietin.

Preferred methods include embodiments wherein said immune regulatory molecule is vascular endothelial growth factor.

Preferred methods include embodiments wherein said immune regulatory molecule is interleukin-3.

Preferred methods include embodiments wherein said immune regulatory molecule is interleukin-4.

Preferred methods include embodiments wherein said immune regulatory molecule is interleukin-10.

Preferred methods include embodiments wherein said immune regulatory molecule is interleukin-13.

371. Preferred methods include embodiments wherein said immune regulatory molecule is interleukin-20.

Preferred methods include embodiments wherein said immune regulatory molecule is interleukin-22.

Preferred methods include embodiments wherein said immune regulatory molecule is interleukin-27.

Preferred methods include embodiments wherein said immune regulatory molecule is interleukin-38.

Preferred methods include embodiments wherein said artificial organ is rendered to possess enhanced regenerative activity by seeding said organ to be bioprinted with a higher number of regenerative cells than a typical homologous organ would possess.

Preferred methods include embodiments wherein said regenerative cells would be mesenchymal stem cells.

Preferred methods include embodiments wherein said mesenchymal stem cells would be umbilical cord derived.

Preferred methods include embodiments wherein said mesenchymal stem cells would be umbilical cord blood derived.

Preferred methods include embodiments wherein said mesenchymal stem cells would be peripheral blood derived.

Preferred methods include embodiments wherein said mesenchymal stem cells would be bone marrow derived.

Preferred methods include embodiments wherein said mesenchymal stem cells would be adipose tissue derived.

Preferred methods include embodiments wherein said mesenchymal stem cells would be selected for enhanced expression of VEGF receptor.

Preferred methods include embodiments wherein said mesenchymal stem cells would be selected for enhanced expression of FGF-1 receptor.

Preferred methods include embodiments wherein said mesenchymal stem cells would be selected for enhanced expression of FGF-2 receptor.

Preferred methods include embodiments wherein said mesenchymal stem cells would be selected for enhanced expression of FGF-5 receptor.

Preferred methods include embodiments wherein said mesenchymal stem cells would be selected for enhanced expression of follistatin receptor.

Preferred methods include embodiments wherein said mesenchymal stem cells would be selected for enhanced expression of endoglin receptor.

Preferred methods include embodiments wherein said mesenchymal stem cells would be selected for enhanced expression of angiopoietin receptor.

Preferred methods include embodiments wherein said mesenchymal stem cells would be selected for enhanced expression of estradiol receptor.

Preferred methods include embodiments wherein said mesenchymal stem cells would be selected for enhanced expression of CD90.

Preferred methods include embodiments wherein said mesenchymal stem cells would be selected for enhanced expression of CD56.

Preferred methods include embodiments wherein said mesenchymal stem cells would be selected for enhanced expression of DAF.

### DETAILED DESCRIPTION OF THE INVENTION

The invention provides the utilization of various immune modulatory cellular and genetic features to the process of tissue engineering leveraging advances in 3D cellular printing technology. In some embodiments the invention provides the utilization of conventional biocompatible customized 3D printed constructs with a customized scaffold concept which is seeded with various immune modulatory and/or regenerative cells in order to facilitate longer retention of the engineered graft, as well as to provide ability for the engineered organ to regenerate. In some embodiments the invention provides creation of organs which function as conventional organs, however, have added therapeutic abilities such as the ability to regenerate. For example, the liver, due to its existing high concentration of endogenous stem cells possesses ability to regenerative. In embodiment artificially generated organs are created through seeing with a higher number of endogenous stem cells in order to increase regenerative activity. This is particularly useful in organs such as pancreas in a diabetic, in which said organ is constantly under immunological attack.

As used herein, “allograft” means an organ or tissue derived from a genetically non-identical member of the same species as the recipient.

As used herein, “bio-ink” means a liquid, semi-solid, or solid composition comprising a plurality of cells. In some embodiments, bio-ink comprises cell solutions, cell aggregates, cell-comprising gels, multicellular bodies, or tissues. In some embodiments, the bio-ink additionally comprises support material. In some embodiments, the bio-ink additionally comprises non-cellular materials that provide specific biomechanical properties that enable bioprinting.

As used herein, “bioprinting” means utilizing three-dimensional, precise deposition of cells (e.g., cell solutions, cell-containing gels, cell suspensions, cell concentrations, multicellular aggregates, multicellular bodies, etc.) via methodology that is compatible with an automated, computer-aided, three-dimensional prototyping device (e.g., a bioprinter).

As used herein, “cartridge” means any object that is capable of receiving (and holding) a bio-ink or a support material.

As used herein, a “computer module” means a software component (including a section of code) that interacts with a larger computer system. In some embodiments, a software module (or program module) comes in the form of a file and typically handles a specific task within a larger software system. In some embodiments, a module may be included in one or more software systems. In other embodiments, a module may be seamlessly integrated with one or more other modules into one or more software systems. A computer module is optionally a stand-alone section of code or, optionally, code that is not separately identifiable. A key feature of a computer module is that it allows an end user to use a computer to perform the identified functions.

As used herein, term “engineered,” when used to refer to tissues and/or organs means that cells, cell solutions, cell suspensions, cell-comprising gels or pastes, cell concentrates, multicellular aggregates, and layers thereof are positioned to form three-dimensional structures by a computer-aided device (e.g., a bioprinter) according to computer code. In further embodiments, the computer script is, for example, one or more computer programs, computer applications, or computer modules. In still further embodiments, three-dimensional tissue structures form through the post-printing fusion of cells or multicellular bodies similar to self-assembly phenomena in early morphogenesis.

As used herein, “implantable” means biocompatible and capable of being inserted or grafted into or affixed onto a living organism either temporarily or substantially permanently.

As used herein, “organ” means a collection of tissues joined into structural unit to serve a common function. Examples of organs include, but are not limited to, skin, sweat glands, sebaceous glands, mammary glands, bone, brain, hypothalamus, pituitary gland, pineal body, heart, blood vessels, larynx, trachea, bronchus, lung, lymphatic vessel, salivary glands, mucous glands, esophagus, stomach, gallbladder, liver, pancreas, small intestine, large intestine, colon, urethra, kidney, adrenal gland, conduit, ureter, bladder, fallopian tube, uterus, ovaries, testes, prostate, thyroid, parathyroid, meibomian gland, parotid gland, tonsil, adenoid, thymus, and spleen.

As used herein, “patient” means any individual. The term is interchangeable with “subject,” “recipient,” and “donor.” None of the terms should be construed as requiring the supervision (constant or otherwise) of a medical professional (e.g., physician, nurse, nurse practitioner, physician's assistant, orderly, hospice worker, social worker, clinical research associate, etc.) or a scientific researcher.

As used herein, “tissue” means an aggregate of cells. Examples of tissues include, but are not limited to, connective tissue (e.g., areolar connective tissue, dense connective tissue, elastic tissue, reticular connective tissue, and adipose tissue), muscle tissue (e.g., skeletal muscle, smooth muscle and cardiac muscle), genitourinary tissue, gastrointestinal tissue, pulmonary tissue, bone tissue, nervous tissue, and epithelial tissue (e.g., simple epithelium and stratified epithelium), endoderm-derived tissue, mesoderm-derived tissue, and ectoderm-derived tissue.

As used herein, “xenograft” means an organ or tissue derived from a different species as the recipient.

In one embodiment the invention teaches to preparation and application of a robust, porous, three dimensional device for extra-hepatic delivery of islets of Langerhans together with regenerative cells, for treatment of patients with type 1 diabetes, and to a process of producing patient-specific devices using 3D Bioprinting with biocompatible hydrogel inks. The novel approach disclosed herein ensures the islets' viability through the use of a 3D Bioprinted porous structure. The presence of autologous cells isolated as stromal vascular fraction during liposuction provides enhanced viability of the islets, reduces inflammatory immune response, and increases productivity of insulin and its delivery through vasculature developed in the pores of the 3D Bioprinted scaffolding device. Various stem cell types may be utilized as part of this combination during organ printing. In some embodiments islets are 3D printed with dedifferentiated cells to enhance growth in situ. Said dedifferentiated cells can be isolated from mesenchymal stem cells. In one embodiment the invention teaches phenotypically defined MSC which can be isolated from the Wharton's jelly of umbilical cord segments and defined morphologically and by cell surface markers for use directly in bioprinting, or for use to dedifferentiate through means disclosed in the invention, as well as other means such as treatment with HDAC inhibitors which are known in the art.

By dissecting out the veins and arteries of cord segments and exposing the Wharton's jelly, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

The dedifferentiated state of the treated cell, which in the current invention is a mesenchymal stem cell, can be verified by increased expression of one or more genes selected from the group consisting of alkaline phosphatase (ALP), OCT4, SOX2, human telomerase reverse transcriptase (hERT) and SSEA-4. That is, the somatic cells introduced with the reprogramming gene are treated with the functional peptide, and then an initial process in which a colony is generated in the dedifferentiation process is observed through alkaline phosphatase staining (AP staining), and furthermore, expression of Oct4 is verified by immunofluorescence (IF) using an Oct4 antibody. Finally, the MET degree in the dedifferentiation process of the somatic cells is verified by flow cytometry (FACS) using antibodies of CD56 as a marker of human umbilical cord mesenchymal stem cells. Mesenchymal stem cells and an epithelial cell adhesion molecule (EPCAM) as a marker of the epithelial cell. In one embodiment dedifferentiation of mesenchymal stem cells is accomplished by addition of epigenetic modifiers such as DNA demethylating agents, HDAC inhibitors, histone modifiers; and cell cycle manipulation and pluripotent or tissue specific promoting agents such as helper cells which promote growth of pluripotent cells, growth factors, hormones, and bioactive molecules. Examples of DNA methylating agents include 5-azacytidine (5-aza), MNNG, 5-aza, N-methl-N′-nitro-N-nitrosoguanidine, temozolomide, procarbazine, et al. Examples of methylation inhibiting drugs agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA. Examples of histone deacetylase (“HDAC”) inhibitor is selected from a group consisting of hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Preferred examples of histone modifiers include PARP, the human enhancer of zeste, valproic acid, and trichostatine. Particular constituents that the inventors utilize in a preferred media in order to facilitate RNA transformation and dedifferentiation of the RNA comprising target cells into pluripotent cells include trichostatine, valproic acid, zebularine and 5-aza.

In some embodiments, devices, systems, and methods for fabricating tissues and organs are utilized as means of assembling 3 dimensional biological structures, in which mesenchymal stem cells are utilized as either structural or functional materials. In some embodiments, the devices are bioprinters. In some embodiments, the methods comprise the use bioprinting techniques. In further embodiments, the tissues and organs fabricated by use of the devices, systems, and methods described herein are bioprinted. In some embodiments, bioprinted cellular constructs, tissues, and organs are made with a method that utilizes a rapid prototyping technology based on three-dimensional, automated, computer-aided deposition of cells, including cell solutions, cell suspensions, cell-comprising gels or pastes, cell concentrations, multicellular bodies (e.g., cylinders, spheroids, ribbons, etc.), and support material onto a biocompatible surface (e.g., composed of hydrogel and/or a porous membrane) by a three-dimensional delivery device (e.g., a bioprinter). A number of methods are available to arrange cells, multicellular aggregates, and/or layers thereof on a biocompatible surface to produce a three-dimensional structure including manual placement, positioning by an automated, computer-aided machine such as a bioprinter is advantageous. Advantages of delivery of cells or multicellular bodies with this technology include rapid, accurate, and reproducible placement of cells or multicellular bodies to produce constructs exhibiting planned or pre-determined orientations or patterns of cells, multicellular aggregates and/or layers thereof with various compositions. Advantages also include assured high cell density, while minimizing cell damage. In some embodiments, methods of bioprinting are continuous and/or substantially continuous. A non-limiting example of a continuous bioprinting method is to dispense bio-ink from a bioprinter via a dispense tip (e.g., a syringe, capillary tube, etc.) connected to a reservoir of bio-ink. In further non-limiting embodiments, a continuous bioprinting method is to dispense bio-ink in a repeating pattern of functional units. In various embodiments, a repeating functional unit has any suitable geometry, including, for example, circles, squares, rectangles, triangles, polygons, and irregular geometries. In further embodiments, a repeating pattern of bioprinted function units comprises a layer and a plurality of layers are bioprinted adjacently (e.g., stacked) to form an engineered tissue or organ. In various embodiments, 2, 3, 4, 5, 6, 7, 8, 9, 10, 11, 12, 13, 14, 15, or more layers are bioprinted adjacently (e.g., stacked) to form an engineered tissue or organ. Polymer solutions are shear thinning, meaning the viscosity is decreased with increased shear rate. In order to provide high printing fidelity, which is typically required when one needs to produce a porous structure, the polymer solutions sometimes do not have sufficient shear thinning properties. In contrast to polymer solutions, nanofiber dispersion can perform better as a shear thinning bioink because the fibril can be oriented in the flow and thus exhibit low viscosity at high shear rates. When shear forces are removed, the nanofibril dispersion can relax to high viscosity which provides high printing fidelity.

In some embodiments the process of bioprinting is facilitated by cellulose nanofibrils (CNF), which can be produced by bacteria or isolated from primary or secondary cell walls of plants, are usually around 8-10 nm in diameter and can be up to a micrometer or more long. They are hydrophilic and therefore bind water to their surfaces. They form hydrogels already at very low solid content (0.5-4% by weight). The hydrophilic nature of the CNF surfaces covered by water prevent them from protein adsorption and make them bioinert, which is relevant to biocompatibility. Nanocellulose biomaterials are not biodegradable in the human body, which is a prerequisite for use as a permanent delivering cell vehicle for a long-lasting, long-performing biomedical device. Alginate is a commonly used biopolymer for islet encapsulation. It has been used for immunoprotection of transplanted allogeneic islets from the immune response attack after transplantation. The encapsulation process and delivery process of islets has, however, not yet been well designed. Islets are typically embedded in alginate beads or mixed in bulk alginate hydrogels, and injected subcutaneously or into the peritoneal cavity The problems related to islet transplantation remain unsolved, which are what is addressed in this patent application. First, a large portion of transplanted islets are lost by attack of the immune system. Second, there is lack of oxygenation and nutrient delivery negatively affecting the islets' survival. Overall, an urgent need exists for innovative solutions which would provide efficient and successful, biocompatible use of transplanted islets. There are effectively two major challenges; immune response and lack of vascularization. For utilization and enhancement of islet viability, stem cells are increased in number when generating the hybrid organ. In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

For generation of stem cells useful for creation of hybrid organs and/or artificial pancreas with enhanced regenerative potential, in some embodiments, host cell enucleation for the generation of hybrid stem cells according to the teachings of the present invention can be conducted using a variety of means. In a non-limiting example, ADSCs are plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention donor organs are utilized a the basis for bioprinting. In one embodiment donor organs are retrieved and frozen at −80° C. or less. Organs may be used individually or pool and then decellularized using a gentle immersion process, and handled in a biosafety cabinet throughout the purification process to maintain sterility. After thawing tissue overnight at 4° C., tissue are minced into pieces ˜1 cm3 in size. Minced tissue are rinsed with sterile water or other liquids suitable to maintaining osmolarity to remove excess blood and fluids. Next, tissue is blended in sterile water or other fluid to create a homogenous slurry. Soluble and insoluble portions of tissue were separated by centrifugation. In one embodiment centrifugation is performed at 3,000 rcf and 40° C. Following centrifugation, the supernatant is discarded, new sterile water is added in a 1:4 pellet to water ratio, and the pellet is dispersed and mixed thoroughly. This slurry is centrifuged again, the water is exchanged, and water washes continued until the supernatant had minimal color. Following water washes, the tissue is then washed three times in isopropyl alcohol, following the same procedure. Next the tissue is washed in 3 M NaCl; for this and the remaining washes, centrifugation is performed at 4° C. Tissue is then washed five times with 40 mg/ml sodium deoxycholate (SDC, Sigma Aldrich); with each SDC wash, the tissue is placed on an orbital shaker for at least 18 h before centrifugation. If after five SDC washes the SDC supernatant has color indicative of incomplete washing, additional SDC washes are performed until there is no additional color in the supernatant. Following SDC washes, tissue is washed again with 3 M NaCl, then with 100 U/ml DNase to aid DNA removal, and next with PBS Finally, the tissue preparation is washed ten times in sterile water. Before each new washing solution, the tissue is washed twice in sterile water to rinse out the old solution. The entire process was ˜10 days in duration with the majority of time taken by the SDC wash. The resulting insoluble material, mainly composed of extracellular matrix protein components, is frozen in sterile water and lyophilized then stored at −20° C. until use. Prior to use in scaffold fabrication or submission for mass spectrometry, lyophilized decellularized cardiac tissue was solubilized in sterile 0.1 M acetic acid at a concentration of 5 mg/ml. After sitting in acetic acid overnight at 4° C., the decellularized tissue is homogenized for 5 min. The solution is kept on ice during homogenization to prevent denaturation at high temperature. Porous ECM scaffolds is fabricated from decellularized cardiac tissue using a sacrificial polycaprolactone (PCL) porous scaffold as a template. The PCL scaffold is generated as a template for the ECM protein and later dissolved away. To create the sacrificial porous scaffold, PCL was dissolved in acetone at 50° C. at a concentration of 0.15 g/ml. Next, water was added dropwise to 8% of the total volume. The PCL/acetone/water solution was then mixed with the porogen NaCl, sieved to select for salt crystals between 425 and 500 um (for stem cell and primary cardiac cell ECM scaffolds) or 35° C. to create a uniform saturated salt suspension. The mixture is placed at −20° C. to solidify, and the salt porogen is subsequently removed by immersion in excess water. The resulting PCL foam exhibits a welldefined pore structure with larger pores formed by the salt porogen and smaller pores formed by water. These foams were cut to a thickness of 0.65-0.7 mm (stem cell and primary cardiac scaffolds) or 0.3 mm (for iPSC cardiomyocyte scaffolds) using a Centaur Deli Slicer. Sliced porous PCL scaffolds are cut with biopsy punches into 3 mm diameter slices intended for further studies. Decellularized and solubilized cardiac ECM was then coated onto these porous PCL scaffolds. PCL scaffolds were immersed in a turbid solution of ECM solubilized in 0.1 M acetic acid. The PCL foam and solution were placed in a vacuum chamber to remove gas pockets and facilitate full penetration of the solution into the PCL pores. Once all air bubbles were eliminated, scaffolds were air dried in a biosafety cabinet, forming a single, contiguous coating. This process was repeated to create a total of five ECM coats. Following coating, the bulk PCL matrix was removed by dissolution in 95% acetone at 40° C. for 2 days. Acetone is replaced twice daily. Scaffolds were then transferred into 95% ethyl alcohol and then slowly exchanged into sterile water using several serial solutions of decreasing ethanol content (70, 50, and 25% ethyl alcohol). Finally, the PCLfree scaffolds were washed 10 times in sterile water. The resulting scaffolds, comprising porous, templated decellularized cardiac tissue, were frozen in 100% water, lyophilized, sterilized with ethylene oxide gas, and stored at −20° C. until use. Lyophilized and sterilized porous ECM scaffolds are added to a 50 ml conical tube containing cell suspensions of 106 cells/100 I for primary cardiac cells, and 107 cells/100 I for iPSC cardiomyocytes (on our scaffolds), and allowed to rehydrate for 10 min. During this time, a spatula was used to immerse the buoyant scaffolds continuously in the cell slurry. Air bubbles are removed to ensure homogeneous scaffold seeding; the vial cap was loosened and allowed to de-gas in a vacuum chamber for 10 min, a sufficient time to eliminate air bubbles without removing all dissolved oxygen in the cell slurry. Six scaffolds per well is added to a 12-well plate (Nunc). After 12 h non-adherent cells were discarded and adhered cells were cultured in iCell Cardiomyocyte media (cellular dynamics) for both iCell iPSC cardiomyocytes and primary cardiac cells. Media was changed every other day and cells were cultured in a 37 incubator with 5% supplemental CO2 for a maximum of 7 days.

1. A method of bioprinting a three-dimensional organ comprising the steps of: a) obtaining a computer representation of said organ; b) identifying cellular populations comprising said organ utilizing a visualization and/or immunological visualization means; c) utilizing a system for sequentially layer cells upon each other in order to replication said organ needed to be replicated; and d) growing said organ in vitro and/or in vivo.

2. The method of claim 1, wherein said identification of said cellular populations is performed by histological analysis.

3. The method of claim 2, wherein said histochemical analysis is performed using a computer assisted visualization means which incorporates principal component analysis.

4. The method of claim 3, wherein said principal component analysis is performed by a deep learning system.

5. The method of claim 4, wherein said deep learning is programed to identify and exclude organ abnormalities.

6. The method of claim 1, wherein cells to be seeded into said organ are identified based on cell surface markers.

7. The method of claim 1, wherein cells to be seeded into said organ are identified based on molecular pathway analysis.

8. The method of claim 7, wherein said molecular pathway analysis is based on expression of frizzled associated genes.

9. The method of claim 1, wherein said cells in said organ are identified based on endodermal, ectodermal and mesodermal content.

10. The method of claim 1, wherein said bioprinting is performed in a media capable of sustaining cellular viability and activity.

11. The method of claim 1, wherein said cellular populations used for said bioprinting procedures are selected from a group comprising of: group consisting of salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells, Bowman's gland cells, Brunner's gland cells, seminal vesicle cells, prostate gland cells, bulbourethral gland cells, Bartholin's gland cells, Littre gland cells, uterus endometrium cells, goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cells, oxyphil cells, adrenal gland cells, chromaffin cells, Leydig cells, theca interna cells, corpus luteum cells, granulosa lutein cells, theca lutein cells, juxtaglomerular cells, macula densa cells, peripolar cells, mesangial cells, blood vessel and lymphatic vascular endothelial fenestrated cells, blood vessel and lymphatic vascular endothelial continuous cells, blood vessel and lymphatic vascular endothelial splenic cells, synovial cells, peritoneal serosal cells, pleural serosal cells, pericardial cavity serosal cells, squamous cells, columnar cells, dark cells, vestibular membrane cells, stria vascularis basal cells, stria vascularis marginal cells, cells of Claudius, cells of Boettcher, choroid plexus cells, arachnoid squamous cells, pigmented ciliary epithelium cells, non-pigmented ciliary epithelium cells, corneal endothelial cells, peg cells, respiratory tract ciliated cells, oviduct ciliated cells, uterine endometrial ciliated cells, rete testis ciliated cells, ductulus efferens ciliated cells, ciliated ependymal cells, epidermal keratinocytes, epidermal basal cells, fingernail and toenail keratinocytes, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, stratified squamous epithelium, epithelial basal cells, urinary epithelium cells, inner auditory hair cells of the organ of Corti, outer auditory hair cells of the organ of Corti, basal cells of olfactory epithelium, cold-sensitive primary sensory neurons, heat-sensitive primary sensory neurons, epidermal Merkel cells, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cells, type I hair cell of the vestibular apparatus of the ear, type II hair cell of the vestibular apparatus of the ear, type I taste bud cells, cholinergic neural cells, adrenergic neural cells, peptidergic neural cells, inner pillar cells of the organ of Corti, outer pillar cells of the organ of Corti, inner phalangeal cells of the organ of Corti, outer phalangeal cells of the organ of Corti, border cells of the organ of Corti, Hensen cells of the organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tube cells, kidney collecting duct cells, type I pneumocytes, pancreatic duct cells, non-striated duct cells, duct cells, intestinal brush border cells, exocrine gland striated duct cells, gall bladder epithelial cells, ductus efferens non-ciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, non-epithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, cochlear stellate cells, hepatic stellate cells, pancreatic stellate cells, red skeletal muscle cells, white skeletal muscle cells, intermediate skeletal muscle cells, nuclear bag cells of the muscle spindle, nuclear chain cells of the muscle spindle, satellite cells, cardiomyocytes, nodal cardiomyocytes, Purkinje fiber cells, smooth muscle cells, myoepithelial cells of the iris, myoepithelial cells of the exocrine glands, reticulocytes, megakaryocytes, monocytes, connective tissue macrophages, epidermal Langerhans cells, dendritic cells, microglial cells, neutrophils, eosinophils, basophils, mast cells, helper T cells, suppressor T cells, cytotoxic T cells, natural killer T cells, B cells, natural killer cells, melanocytes, retinal pigmented epithelial cells, oogonia/oocytes, spermatids, spermatocytes, spermatogonium cells, spermatozoa, ovarian follicle cells, Sertoli cells, thymus epithelial cells, and intestinal kidney cells.

12. The method of claim 1, wherein bioprinting comprises three-dimensional printing of a biological organ, organoid, and/or tissue through the layering of living cells using a bioprinter.

13. The method of claim 12, wherein said bioprinter is a three-axis mechanical platform that controls the movements of extruders that deposit layers of living cells in a desired shape.

14. The method of claim 13, wherein said desired shape is acquired by scanning the surface of a desired organ, organoid and/or tissue to generate a surface map for guidance with cell deposition.

15. The method of claim 14, wherein scanning the surface of a desired organ, organoid and/or tissue is achieved using a laser, electron beam, magnetic resonance imaging, microwave, x-ray, computed tomography, or a combination thereof.

16. The method of claim 1, wherein said three dimensional organ is manufactured in a manner to possess reduced immunogenicity as compared to a wild type organ.

17. The method of claim 16, wherein said reduced immunogenicity is accomplished by inhibition of immunogenic epitopes.

18. The method of claim 17, wherein said reduction in HLA expression is achieved by gene editing to remove said HLA molecules from cells to be manufactured as part of the bioprinted organ.

19. The method of claim 17, wherein said reduction of immunogenic molecules is accomplished by administration of agents capable of inducing the process of RNA interference.

20. The method of claim 19 wherein said reduction of immunogenicity of immunogenic epitopes is accomplished by engineering the organ with immune regulatory molecules.
